(19) United States
(12) Patent Application Publication
(10) Pub. No.: US 2023/0166135 A1
(43) Pub. Date: Jun. 1, 2023

(54) RESPIRATOR MASK WITH A FILTER

(71) Applicant: WOCO Industrietechnik GmbH, Bad Soden-Salmünster (DE)

(72) Inventor: Fritz Schmitt, Rosport (LU)

(73) Assignee: WOCO Industrietechnik GmbH, Bad Soden-Salmünster (DE)

(21) Appl. No.: 17/997,322

(22) PCT Filed: Apr. 27, 2021
(86) PCT No.: PCT/DE2021/100379

§ 371 Date: Oct. 27, 2022

(30) Foreign Application Priority Data
Apr. 27, 2020 (DE) 102020111399.4
Dec. 1, 2020 (DE) PCT/DE2020/101015

(51) Int. Cl. A62B 23/02; A62B 7/10; A61L 9/20; A61L 9/014; B01D 46/00; B01D 53/047

(52) U.S. Cl. CPC A62B 23/02; A62B 7/10; A61L 9/20; A61L 9/014; B01D 46/0036; B01D 46/0028; B01D 46/0032; B01D 53/047; A61L 2209/14; A61L 2209/12; A61L 2209/22; A61L 2209/111; B01D 2253/102; B01D 2253/108; B01D 2279/40; B01D 2256/12; B01D 2259/4541; B01D 2259/4533

(57) ABSTRACT

The disclosure relates to a respirator mask including a mask body and at least one filter for filtering a fluid (e.g. air) flowing through the mask and/or the mask body. The at least one filter may include one or more of: an Ultra-Violet (UV) filter and/or UV emitter, a filter fleece, and/or an electrical and/or electrostatic filter.

### CROSS REFERENCE TO RELATED APPLICATIONS

This is a U.S. National Stage Patent Application of PCT/DE2021/100379, filed Apr. 27, 2021, which claims priority to German Patent Application No. 102020111399.4, filed Apr. 27, 2020, and International Application No. PCT/DE2020/101015, filed Dec. 1, 2020. Each of these applications is incorporated herein by reference in its entirety.

### BACKGROUND

### Field

The disclosure is based on a respirator mask for respiratory protection of a mask wearer, with a mask body and at least one filter for filtering a fluid, such as air, flowing through the mask and/or the mask body.

### Related Art

Filtering half masks are mask and filter in one. In the case of known half masks, usually the filter cannot be replaced. Rather, these half masks are disposed completely after use, or when the filter is exhausted. Half masks are usually relatively light and comfortable to wear and have a relatively large filter area and are relatively hygienic. On the downside, common half masks are overall somewhat more expensive to use compared to full masks and are usually not suitable for filtering gaseous pollutants. Half masks are often used as work protection, e.g. in the medical field to protect against infections, but also e.g. when working with dust, wood, fiberglass or concrete, comprise a mask body, usually made of rubber or silicone, and encompass the mouth and nose area of a mask wearer. One or two filter cartridges may be attached to the mask body in some embodiments.

A half mask of the type mentioned above is known from DE 40 17 336 C1. The known half mask has a half mask body with a sealing rim, which is inserted into a filter holder. In the mouth area, a filter is attached to the filter holder. The filter holder extends from the mouth area to the cheek area of the half-mask body and rests freely on the half-mask body. Inhalation takes place through an inhalation valve, and exhalation takes place through an exhalation valve buttoned into the half-mask body in the chin area. A strap is fastened in an eyelet of the filter holder, with which the half-mask can be fastened to the head of a device wearer.

In the known half mask, the tightness between the oral cavity and the environment is determined by the geometry and rigidity of the sealing rim, the resilience of the half mask body and the lateral support of the half mask body by the filter holder. A soft mask body or soft sealing rim increases the wearing comfort, but worsens the mechanical stability, while a high stiffness of the mask body or sealing rim is perceived as uncomfortable when wearing the half mask.

The filter holder, which rests against the half-mask body in the cheek area, only provides a certain lateral stabilization of the half-mask body, while there is no direct interaction between the filter holder and the sealing rim, which is decisive for the tightness of the half-mask.

A half mask known from GB-PS 761 263 consists of a flexible half mask body into which a filter holder with a filter is inserted in the mouth area. In the area of the sealing rim of the half-mask body, a wire filament is vulcanized into the mask body to give the sealing rim an appropriate rigidity. The wire filament can be roughly adapted to the facial contour of the mask wearer.

During the COVID 19 pandemic, in public mainly homemade everyday masks or medical hygiene masks, e.g., surgical masks or FFP masks (“filtering face piece” masks) of protection class FFP 1 or FFP 2 are worn. Particle-filtering half masks or FFP masks, depending on the design, protect against the inhalation of particles and aqueous or oily aerosols. They are mostly made entirely of nonwoven fabric with rubber straps and a formable nose clip to optimize the alignment to the face.

Standardized masks with CE marking can protect against respirable dusts and liquid mists within their respective area of application when used properly. In addition to the supporting filter material, they can comprise layers, e.g., with a meltblown fleece, with an electrostatic material. Small dust particles and liquid droplets can be bound in the filter by electrostatic forces. However, the electrostatic effect is lost very quickly due to exhaled humid air and dust accumulation. Tests have shown that after wearing the mask for two hours, the static charge is already lost, partly due to moisture that collects in the mask fabric. Even by drying the mask, e.g., in an oven, the mask function cannot be restored.

### BRIEF DESCRIPTION OF THE DRAWINGS/FIGURES

The accompanying drawings, which are incorporated herein and form a part of the specification, illustrate the embodiments of the present disclosure and, together with the description, further serve to explain the principles of the embodiments and to enable a person skilled in the pertinent art to make and use the embodiments.

FIG. 1 is a mask according to an exemplary embodiment of the disclosure in a donned state.

FIG. 2 is the mask of FIG. 1 in a plan view, according to an exemplary embodiment of the disclosure.

FIG. 3A-3B show a mask according to exemplary embodiments of the disclosure.

FIG. 4 is a filter according to an exemplary embodiment of the disclosure.

FIG. 5 is a mask with the filter of FIG. 4 and an oxygen generator, according to an exemplary embodiment of the disclosure.

FIG. 6 is a mask with the filter of FIG. 4, according to an exemplary embodiment of the disclosure.

The exemplary embodiments of the present disclosure will be described with reference to the accompanying drawings. Elements, features and components that are identical, functionally identical and have the same effect are—insofar as is not stated otherwise—respectively provided with the same reference character.

### DETAILED DESCRIPTION

In the following description, numerous specific details are set forth in order to provide a thorough understanding of the embodiments of the present disclosure. However, it will be apparent to those skilled in the art that the embodiments, including structures, systems, and methods, may be practiced without these specific details. The description and representation herein are the common means used by those experienced or skilled in the art to most effectively convey the substance of their work to others skilled in the art. In other instances, well-known methods, procedures, components, and circuitry have not been described in detail to avoid unnecessarily obscuring embodiments of the disclosure. The connections shown in the figures between functional units or other elements can also be implemented as indirect connections, wherein a connection can be wireless or wired. Functional units can be implemented as hardware, software or a combination of hardware and software.

An object of the disclosure to provide a mask that overcomes these disadvantages and whose electrostatic filtering effect lasts longer. The filtering effect of the mask according to the disclosure can be improved.

The present disclosure is characterized in that the at least one filter comprises at least one or more of activated carbon, Ultra-Violet (UV) filter and/or UV emitter, filter fleece, electrical and/or electrostatic filter, membrane filter and/or particle filter. For example, it may be provided that the filter comprises an electrical and/or electrostatic filter. Additionally, the filter may comprise, for example, a filter fleece. Combinations of activated carbon, UV filter and/or UV emitter, filter fleece, electrical and/or electrostatic filter, membrane filter and/or particle filter may also be possible. The filter fleece may be a non-woven fabric. The filter fleece can be set up to filter pollutants and/or viruses from a fluid.

The mask may comprise at least one filter holder, wherein at least one filter may be received in the filter holder, wherein the filter holder may be and/or substantially arranged at least in the mouth region and/or in the nose region of a mask wearer in a donned state of the mask.

The filter can be detachably connected to the mask and/or the mask body, such as to the filter holder. This allows a filter to be replaced quickly, for example when the filter effect is too low due to use or in order to regenerate the filter.

The filter and/or the electrical filter and/or electrostatic filter may comprise a grid. The grid may comprise a metal, such as copper, brass, silver, gold and/or combinations thereof. The grid may also comprise corresponding alloys, for example an alloy comprising copper, brass, silver and/or gold. The grid may be or may get powered. The grid may be or become electrostatically charged. The grid may be or become electrostatically charged by a power supply.

The grid may be arranged between a first layer and a second layer, wherein the first and/or the second layer may comprise a fleece. The grid can be sandwiched between the two layers. The two layers may support and/or fix the grid. The fleece may be an or the filter fleece. The fleece may comprise a filtering effect.

The first and/or the second layer may be or comprise a meltblown fleece. The meltblown fleece may comprise a plastic, such as polypropylene. The filter fleece may be or comprise a meltblown fleece.

The filter may comprise a molecular sieve. The molecular sieve can comprise zeolites or activated carbon. The molecular sieve may be arranged in front of or behind the grid in the direction of flow through the filter. The molecular sieve may be supported, held, or enclosed by the first, second, or a further layer.

The UV emitter can be arranged in the filter in such a way that an or the filter fleece can be illuminated by the UV emitter. This way pollutants or viruses present or filtered on or in the filter fleece can be deactivated or decomposed by the UV light. The filter fleece can be or become disinfected by illumination with the UV emitter. This can increase the reusability of the mask. The filter fleece may have a fluorescent substance with which the filter fleece is impregnated. The fluorescent material may be adapted to be activated by the UV emitter. The emitted wavelength of the fluorescent material may be 250-400 nm, which has been shown to be harmful to many viruses.

The mask and/or filter may include an energy storage, such as a replaceable battery, and a controller. The energy storage may be connected to the electrical filter and/or electrostatic filter and/or the grid for power supply, wherein the controller may be arranged to control or regulate the power supply. For example, the controller may activate or deactivate a power supply of the grid. Alternatively, or additionally, the energy storage may be connected to the UV emitter for power supply, wherein the controller may be adapted to control or actuate the UV emitter.

The mask and/or filter may comprise a solar cell that may be connected to the energy storage and/or the controller. The energy storage can be chargeable by the solar cell.

The mask and/or the filter can comprise a flow sensor that can be adapted to detect fluid to be filtered flowing in through the mask and/or the filter and/or fluid flowing out of the mask and/or the filter.

The flow sensor may be connected to an or the controller so that upon detection of fluid flowing through the mask and/or filter, such as fluid flowing in and/or out, the controller may control the power supply to the grid and/or the electrical filter. Alternatively, or additionally, when detecting fluid flowing through the mask and/or filter, such as fluid flowing in and/or out, the controller can control the UV emitter so that the filter fleece can be or get illuminated by the UV emitter.

The flow sensor can comprise a pivot plate and a contact, which is pivotable about an axis of rotation into a flow-through position when flow passes through the filter, such that in the flow-through position the pivot plate can contact the contact. If the contact is contacted by the pivot plate, it may be provided that the flow sensor transmits a signal to the controller that indicates a flow-through. The flow-through may be or comprise a flow of a fluid into the filter or mask. It may be provided that the flow sensor comprises a second contact arranged in such a way that it is contacted by the pivot plate in the event of a flow-through of the filter in the opposite direction (e.g., a flow out). In this way, the flow sensor can easily detect different flow directions and communicate them to the controller.

The mask and/or the mask body may at least sectionally comprise a superabsorber. The superabsorber may form at least part of the mask body. The superabsorber may be adapted to absorb liquid.

The superabsorber can be arranged, on or in an inner side of the mask, in such a way that moisture located inside the mask in a donned state of the mask can be absorbed or can be absorbable by the superabsorber. The superabsorber can be arranged, for example, at those points on the inner side of the mask where condensate collects and/or exhaled air preferentially condenses when the mask is put on. For example, the superabsorber may be arranged in an area that may be at, near, or close to an area of the mouth or nose in a donned state of the mask.

The mask can comprise an oxygen generator, whereby the oxygen generator can be fluidically connected to an oxygen admixing unit (oxygen admixer), which can be adapted to enrich fluid flowing through the mask with oxygen. It may be provided that the mask comprises a plurality of oxygen generators, for example in order to be able to provide oxygen continuously. If the oxygen generators need to be regenerated, the plurality of oxygen generators may be controlled or operated cyclically in such a way that when at least one oxygen generator is regenerated, at least one of the remaining oxygen generators does not regenerate respectively does provide oxygen. For example, if one of the oxygen generators is regenerated, the remaining oxygen generator(s) may provide oxygen.

The oxygen generator may be adapted to provide or generate oxygen by means of a pressure swing adsorption process or electrolysis. The oxygen generator may be suitably designed, dimensioned and/or constructed.

The filter can be fluidically connected to the oxygen-admixing unit such that air entering the mask is filtered by the filter and supplied to the oxygen-admixing unit. Depending on the mode of operation of the oxygen generator, alternatively or additionally, an or the filter can be fluidically connected upstream of the oxygen generator, for example if the oxygen generator generates oxygen from air.

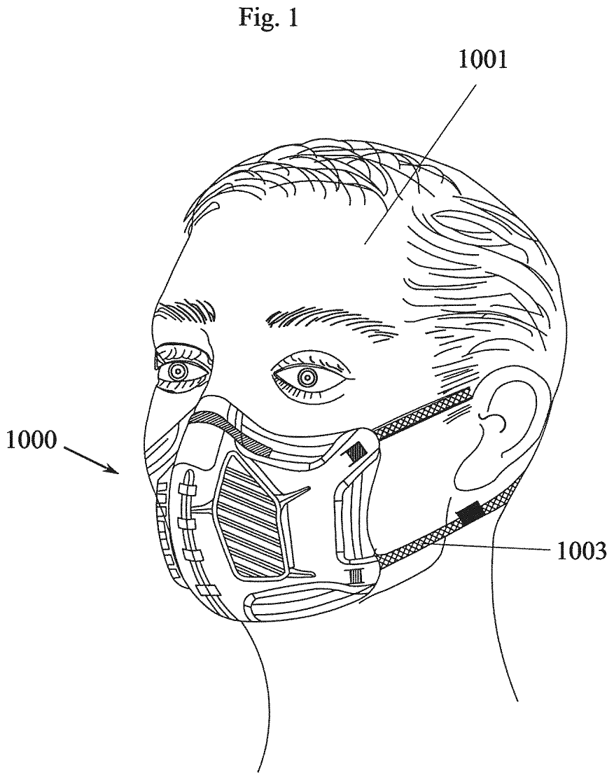

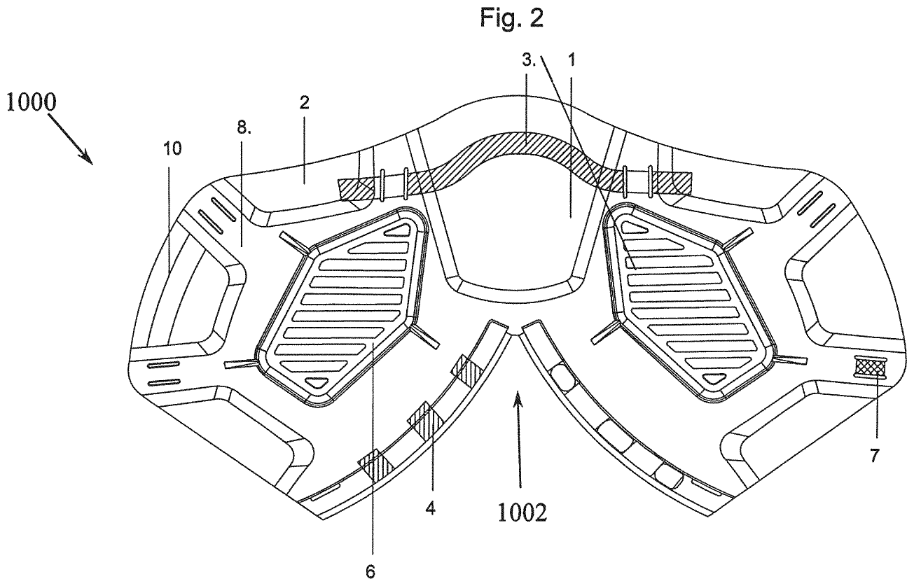

FIGS. 1 and 2 show an embodiment of a mask 1000 according to the disclosure. The mask 1000 may be a half mask. The mask 1000 comprises a mask body 1. FIG. 1 shows a mask wearer 1001 with an embodiment of the mask 1000 in a donned state. FIG. 2 shows the embodiment of the mask 1000 of FIG. 1 in a state before donning by the mask wearer 1001 or in a discarded state. The mask 1000 may be made of or comprise a material that is washable, for example washable in a washing machine, and/or disinfectable. The mask 1000 may made of or comprise a material that is biodegradable or recyclable.

The material may be or comprise a soft plastic with shore hardness of 20-80. The mask 1000 or mask body 1 may comprise different zones, for example first zone 2, reinforced zone 8 and/or flexible zone 10, with different material thickness or shore hardness, so that a good fitting and/or a good fit of the mask 1000 on the face of the mask wearer 1001 in the donned state can be achieved. The material may comprise a memory effect, particularly with respect to its shape. The material of the mask 1000 may conform to the face of the mask wearer 1001 when worn. It may be provided to perform a curing of the mask 1000 by UV radiation.

As shown in FIG. 2, the mask 1000 may have a butterfly shape in its discarded state and/or original state. The mask 1000 may have a suitable cutout, for example a V-shaped cutout 1002 in FIG. 2. However, other shapes of the mask 1000 are conceivable. In its discarded state and/or original state, the mask 1000 may have substantially a two-dimensional shape. In the donned state, see for example FIG. 1, or when the mask 1000 is put on, the two wings of the butterfly shape may be connected to each other, resulting in a three-dimensional shape of the mask 1000 as shown in FIG. 1. The three-dimensional shape of the mask 1000 may be substantially shaped such that it substantially corresponds to a face shape or head shape of a mask wearer 1001. Thus, the mask 1000 may fit particularly tightly against the face of the user 1001. The left “wing” of the mask 1000 may correspond to a left half of the mask. The right “wing” of the mask 1000 may correspond to a right half of the mask. In the embodiment shown in FIG. 2, the left and right mask halves are integrally formed with each other. However, it may also be provided that, when the mask 1000 is in the discarded state, the mask 1000 may comprise a plurality of mask parts 1000 that are separate from each other or at least partially connected to each other. The connection of the respective mask parts may then take place when and/or before the mask 1000 is put on. In at least one embodiment, the connection of the mask halves may be accomplished via a closure 4. In an embodiment not shown, the closure 4 may be or comprise a zipper.

The mask 1000 comprises a mask body 1. The mask body 1 may be multilayered. The mask body 1 may be made of or comprise one or more thermoplastic materials. The mask body 1 may be manufactured by a thermoforming process or by an injection molding process. The mask body 1 may be made of or comprise plastics, silicones, and/or fabrics or a combination thereof. If the mask body 1 is multilayered, each layer may be made of or comprise one or more plastics, silicones and/or fabrics or a combination thereof and one, more or all layers may be made of or comprise the same or different material.

The mask body 1 can be foamed in a mold. The foamed material can contain additives that have an antibacterial effect, e.g., silver ions. The mask body 1 can serve as a holder for filter elements and/or itself be a filter and/or comprise a filter function. The material of the mask body 1 may be porous. By certain folding techniques and cuts, a three-dimensional mask body 1 can be created from a two-dimensional mask body 1.

The mask body 1 may made of or comprise one or more zones, e.g., first zone 2, flexible zone 10. The Shore hardness of different zones 2, 8, 10 may be different or the same. The zones 2, 8, 10 may comprise different material thickness. The zones 2, 8, 10 may comprise different materials. It can be provided that in the first zone 2 the mask 1000 or the mask body 1 can be cut, for example, with scissors.

The mask 1000 and/or the mask body 1 may comprise a shaper 3. The shaper 3 may be made of or comprise a pliable material. The shaper 3 may be made of or comprise a plastic, a metal, or a metal alloy. The shaper 3 may retain its shape and serve to adapt the mask 1000 to the anatomical shape of a user. It may be provided that the shaper 3 is deformable by bending so that the shape of the mask 1000 is adaptable to a facial shape of a mask wearer 1001. The shaper 3 may also at least partially be made of or comprise a plastic material that can be cured by UV light. Alternatively, or additionally, the mask 1000 and/or the mask body 1 may at least partially be made of or comprise a plastic that can be cured by UV light. If the shaper 3 and/or the mask 1000 and/or the mask body 1 comprises such a plastic, an unused mask 1000 can be stored and/or delivered in a light-tight, black cover.

The mask body 1 can have a substantially circumferential support means at least in the cheek area and in the nose area, which can be designed in the cheek area as a first shoulder as a stop for a first end face of a filter holder 6. In the nose region, the support means may be designed as a second shoulder as a stop for a second end face of the filter holder 6. The end faces and the shoulders may be engaged by a strap 1003 in a manner supporting a sealing rim when the mask is in place. The support means may be or comprise the shaper 3. It may also be provided that the support means is of a multi-part design. It is also conceivable that the support means is subsequently attached and/or fastened to the mask body 1.

The mask 1000 or mask body 1 may comprise an at least partially circumferential sealing rim. The sealing rim can be arranged at and/or along the edge of the mask body 1. The mask body 1 may be stiffened by the support means in the region of the sealing rim. For example, the support means may be engaged, e.g., substantially circumferentially, on the mask body 1 with the end faces of the fixed filter holder 6 in a donned state of the mask 1000 due to the pull of the strap. The sealing rim or the mask body 1 can therefore be made of particularly flexible material, for example a flexible elastomer, since the support of the sealing rim can be provided by the filter holder 6 resting against the support means. By attaching a support means to the mask body 1 in a defined manner, it may also be possible to adjust the stiffness of the sealing rim in the respective area of the mask body 1. The stiffness can be influenced, for example, by changing the distance between the support means and the sealing rim, or by a specific geometry of the support means in interaction with the end face of the filter holder 6. Between the support means and the associated end faces of the filter holder 6, one or more adhesive layers can expediently be present, by means of which the sealing rim of the mask body 1 can be additionally fixed in and/or on the filter holder 6, for example if the pull of the strap 1003 is not yet fully effective when the mask 1000 is put on.

The mask 1000 and/or the mask body 1 can comprise at least one filter holder 6. The filter holder 6 may be connected to the mask 1000 and/or the mask body 1. The filter holder 6 may be integrally formed with the mask 1000 and/or mask body 1. The filter holder 6 may also be retrofittable, i.e., be subsequently connected to the mask 1000 or mask body 1. The filter holder 6 may be or comprise a filter chamber. The filter holder 6 may serve to hold one or more, possibly different, filters. For example, one or more filter mats may be received in the filter holder 6. The filter holder 6 may have an outer grid structure. The outer grid structure may be of such a fine mesh that the filter holder 6 can be used as a pre-filter. The filter holder 6 may also be formed as a chamber with a closure. The chamber may receive filters and/or substances in loose form, for example granules, gel beads, fibers, silica, zeolite. The filter may comprise at least one or more of membrane, particle filter, UV filter, biofilter, air filter, gas filter, activated carbon filter, electrostatic filter, electro filter, cyclonic filter, liquid filter, oil-impregnated filter, bag filter, or the like, individually or in combination.

The mask 1000 and/or the mask body 1 may comprise one or more filter holders 6, which may be arranged symmetrically, for example. If the mask 1000 comprises, for example, two mask halves or several mask parts, it can be provided that one or both mask halves or one or more of the mask parts each have a filter holder 6.

Special embodiments of the filter holder 6 may be or comprise a first filter holder 6 for a filter with a screw thread and/or a second filter holder 6 for a respiratory filter with a round thread. The first filter holder 6 and the second filter holder 6 or embodiments thereof may differ, for example, only in the specific connection of the respective types of filters. The filters can be attached directly to or in the connection opening of the filter holder 6.

The support means may be configured in the cheek region of the mask body 1 as a substantially circumferential first shoulder as a stop for a first end face of the filter holder. When the mask 1000 is placed against the face of the mask wearer 1001 with the strap, the support of the sealing rim in the cheek region may be provided by the first shoulder, in that the first end face of the filter holder 6 may abut the first shoulder due to the pull of the strap 1003. The support means in the nose region may be configured as a substantially circumferential second shoulder, which may be bead-shaped, for example, and may abut directly against a second end face of the filter holder 6. In the nose region of the mask body 1, an additional, e.g., bellows-shaped, deformation zone may be provided for adapting the mask body 1 to the nose region of the mask wearer 1001.

In a practical manner, the first shoulder can be designed as an inclined, funnel-shaped surface in the transition area between the sealing rim and the mask body 1. In the region of the first shoulder, the first end face of the filter holder 6 can be designed to correspond to the shoulder, e.g., the inclined funnel-shaped surface.

In the chin region, the mask body 1 can comprise a support lip facing the filter holder as a support means. The support lip can be supported against a third end face of the filter holder 6. The support lip can essentially provide support for the mask body in the chin region in the radial direction.

The filter holder 6 can be designed in such a way that it at least partially surrounds and/or covers and/or overlaps the nose region, the cheek region and the chin region of the mask body 1, see for example the embodiment of the mask 1000 shown in FIG. 1. The filter holder 6 can be designed in one piece. The filter holder 6 may thus be or comprise an outer, stabilizing and supporting shell for the mask body 1, which may be non-rigid. The filter holder 6 may be substantially cylindrical in shape. This may enable the filter holder 6 to be manufactured as a molded part in a particularly cost-effective manner However, the filter holder 6 can also comprise other geometric shapes, e.g., a polygonal or honeycomb-like shape as shown in FIG. 2.

The mask 1000 and/or the mask body 1 may comprise a mounting interface 7 for fastening a fastening band or strap. The fastening band may be at least partially flexible and/or stretchable and/or elastic. The fastening band may be or comprise a cord.

The mask body 1 and/or the mask 1000 may at least partially be made of or comprise solid material, for example in a reinforced zone 8. The mask 1000 and/or the mask body 1 may comprise a flexible zone 10, wherein the flexible zone 10 may be held in shape in a stabilized manner and/or be fixed by the reinforced zone 8. The filter holder 6 may be arranged in the region of the reinforced zone 8. The mask body 1 may also comprise a plurality of reinforced zones 8, which may be arranged symmetrically with respect to an axis of symmetry of the mask 1000, for example. By the fact that the mask body 1 or the mask 1000 may comprise at least one or more reinforced zones 8 and flexible zones 10, the mask 1000 may comprise a high stability and/or a good and tight fit of the mask 1000 on the face of a user 1001 may be ensured, with at the same time high wearing comfort.

As described, the mask 1000 and/or the mask body 1 may comprise a flexible zone 10. This may ensure that the mask 1000 has a better seal in areas that are deformed during speech. The flexible zone 10 may, for example, be hollow and/or inflatable to provide an even better adaptation to the deformations of the mask 1000 and/or the mask body 1 caused by speaking.

The mask 1000 and/or the mask body 1 may comprise at least one inflatable cavity. The inflatable cavity may, for example, be located at the edge and/or along the frame of the mask and/or mask body 1. The sealing rim may be or comprise the inflatable cavity. Alternatively, or additionally to the sealing rim, however, the inflatable cavity may also be provided. By suitably filling or discharging the cavity with a fluid, for example air, gas and/or liquid, the mask 1000 can be individually adapted to the facial shape of the user 1001 in the donned state. The mask 1000 may be adapted to this purpose, for example comprise suitable valves. If the mask 1000 and/or the mass body 1 comprises more than one cavity, the cavities may be fluidically separated from each other and/or filled separately. However, it may also be provided that some cavities are fluidically connected to each other. In one embodiment, the mask 1000 can for example be connected to a compressed air cartridge via a suitable connecting element for filling the at least one cavity.

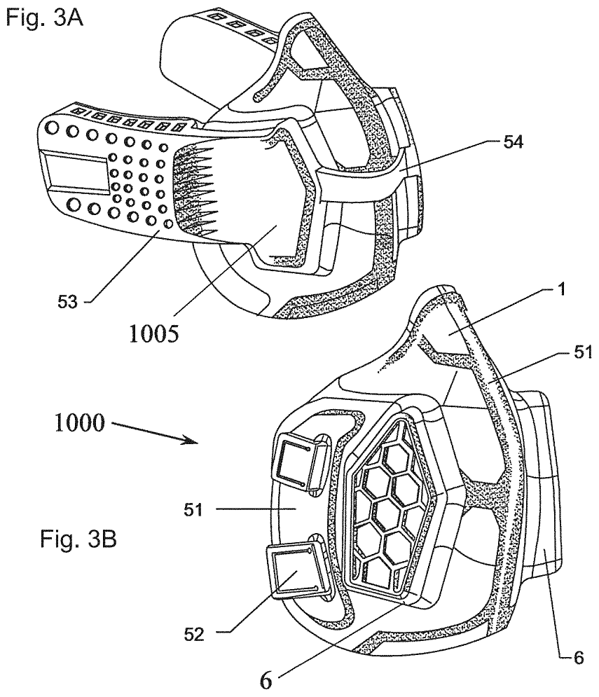

FIGS. 3A and 3B show further embodiments of a mask 1000. The embodiments of a mask 1000 shown in FIGS. 3A and 3B do not comprise a cutout 1002 and do not comprise a closure 4. The mask 1000 and/or the mask body 1 may comprise one or more reinforcement zones 51. The reinforcement zone may be arranged, for example, symmetrically and/or centrally with respect to the mask 1000. The reinforcement zone 51 may be made of or comprise a memory plastic that may be adapted, for example, to memorize an anatomical shape. One or more zones of the mask 1000 and/or the mask body 1, e.g., the reinforcement zone 51, first zone 2, reinforced zone 8, and/or flexible zone 10, may comprise or be made of an electrically conductive plastic such that application of voltage may cause the corresponding zone to change its strength and/or shape.

The mask 1000 may comprise a holder 52 for elastic bands 1003 or the like. The holder 52 may be pivotable. The holder 52 may be arranged and/or attached to the mask body 1. A mask attachment 53 may be placed on the filter holder 6 and/or the filter chamber 6, as shown in FIG. 3A. The mounting interface 7 may be or comprise the holder 52.

The mask attachment 53 may comprise a flexible connecting element 54. For example, if the mask attachment 53 has two or more slip-on attachments 1005, they may be connected to each other by the flexible connecting element 54. In the embodiment shown in FIG. 3, the flexible connecting element 54 connects for example a right slip-on attachment 1005 and a left slip-on attachment 1005. The flexible connecting element 54 may serve as a shaper and/or may be deformable. For example, the flexible connecting element 54 may be made of or comprise a bendable plastic. The mask 1000 and/or the mask body 1 may comprise connecting elements complementary to the slip-on attachment, such that the mask attachment 53 may be attached to the mask 1000 and/or the mask body. The corresponding attachment may be releasable. The slip-on attachment 1005 may be or comprise a mask attachment filter chamber.

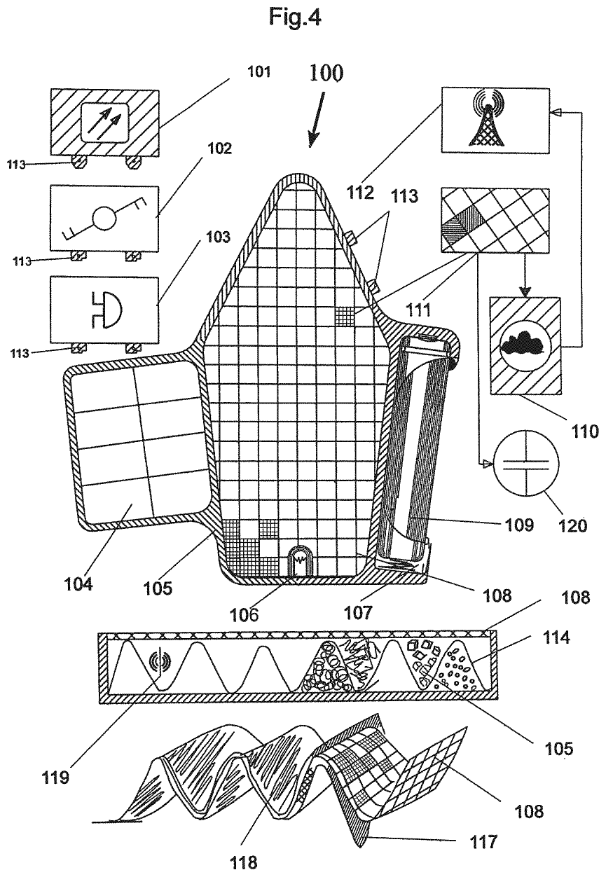

For example, the slip-on attachment 1005 may comprise one or more filters. The filter may be or comprise a filter 100 described below with reference to FIGS. 4 to 6. The slip-on attachment 1005 may comprise an oxygen generator 122 described below. It may alternatively or additionally be provided that the slip-on attachment provides further functions, e.g., headphones, microphone, radio interface, energy storage, or the like.

FIG. 4 shows an embodiment of a filter 100. The filter 100 may be adapted to be received in the filter holder 6 or to be inserted into the filter holder 6. However, it can also be provided that the filter 100 can be slipped onto the filter holder 6. The filter 100 may be detachably connected to the filter holder 6 and/or the mask 1000 and/or mask body 1.

The filter 100 may comprise a UV emitter 106, such as a UV-C LED. The filter may comprise a filter fleece capable of filtering viruses and/or contaminants from a fluid flowing through the filter. The UV emitter 106 may be arranged in the filter 100 such that it can irradiate the filter fleece. Irradiating the filter fleece with the UV emitter 106 may disinfect the filter fleece and/or deactivate or decompose contaminants and/or viruses located in or on the filter fleece. The UV emitter can emit, for example, UV-C light with a wavelength of 100-280 nm. With the UV-C light, for example, biogenic substances or viruses can be decomposed.

The filter 100 may comprise a grid 108. The electrical filter and/or electrostatic filter may comprise the grid 108. The grid 108 may comprise a metal, such as copper, brass, silver, gold, corresponding alloys, and/or combinations thereof. The grid may be or become electrically or electrostatically charged. An electrostatic charge of the grid may comprise a charge separation. With the charge of the grid, particles, contaminants, or viruses present in the fluid flowing through the filter or grid can be separated or held to the grid by Coulomb, dipole, or mirror charge forces. It may be provided that the grid is electrostatically charged during filter manufacture. Alternatively, or additionally, it may be provided that the filter or the grid is electrostatically charged, or brought to a reference charge, at predetermined time intervals or according to other rules, e.g. when an air flow is detected through the filter. This can prevent or reduce a reduction of the charge during operation or a reduction of the filter effect.

The mesh 108 may be arranged between a first layer 118 and a second layer 117. The first layer 118 and/or the second layer 117 may be or comprise an or the filter fleece.

The filter fleece, the first layer 118 and/or the second layer 117 may be or comprise a meltblown fleece. The meltblown fleece may be or comprise a plastic, such as polypropylene (PP). However, the filter fleece, the first layer 118 and/or the second layer 117 may also be or comprise, for example, a polyester, polyamide (PA), PES, PET or combinations thereof. By embedding the grid 108 between the first layer 118 and the second layer 117, respectively the first and/or second layer arranged at the grid 108, the electrostatic charge may last longer respectively the grid 108 may remain electrostatically charged longer. The first layer 118 and/or the second layer 117 may mechanically stabilize or fix the grid 108. The first layer 118 and/or the second layer 117 and/or the filter fleece may comprise a metal powder. Due to the metal powder the first layer 118 and/or the second layer 117 and/or the filter fleece may be electrically conductive. It may also be provided to illuminate the first layer 118, the second layer 117 and/or the filter fleece with the UV emitter 106. The UV emitter may be arranged accordingly. For example, the UV emitter may be arranged between the first layer 118 and the grid 108 or the second layer 117 and the grid 108. However, it may also be provided, for example, that a first UV emitter may illuminate the first layer 118 and a second UV emitter may illuminate the second layer 117; the first UV emitter and/or the second UV emitter may be arranged outside the filter 100 and/or the sandwich structure of the first layer 118, the grid 108 and the second layer 117.

In the production of the meltblown fleece, the plastic, e.g., polypropylene (PP), can first be melted until it can have approximately the consistency of liquid honey. Trough tiny nozzles subsequently a thin filament is formed that can be blown onto a micro-sieve or the grid 108. Thus, the first layer 118 can be formed. In an equivalent manner, the second layer 117 can be formed on the other side of the grid 108. A consistent electrostatic voltage can be generated by the embedded metallic grid sieve 108.

The filter 100 may comprise a molecular sieve 115. The molecular sieve 115 may comprise or be made of activated carbon, carbons, and/or zeolites. The molecular sieve 115 may filter contaminants and/or viruses from the fluid flowing through the filter. For example, the molecular sieve 115 may be received or arranged in a receptacle formed by a layer 114, which may be fleece fabric. The molecular sieve 115 may alternatively or additionally be received or arranged in the filter between two layers of fleece fabric, e.g., the first layer 118 and the second layer 117.

The filter 100 may comprise a controller 111. The controller 111 may be or include a microprocessor or the like. The controller 111 may be connected to the grid 108. The controller 111 may be adapted to cause and/or initiate a charge separation of the grid 108, such that the grid 108 may be electrostatically charged at the instigation of the controller 111. Alternatively, or additionally, it can be provided that the controller 111 controls and/or regulates a power supply to the grid 108. For example, when in case of a flow through the filter 100, the controller 111 may let current flow through the grid 108 so that the grid 108 may act as an electrical filter.

Alternatively, or additionally, the controller 111 may be connected to the UV emitter 106. The controller 111 may be adapted to control and/or actuate, or turn on or turn off, the UV emitter 106.

The filter 100 may comprise an energy storage device 109. The energy storage device 109 may be received in an energy storage receptacle 107. Alternatively, or additionally, the mask 1000 and/or the mask body 1 may comprise an or the energy storage device 109. The energy storage device 109 may be or comprise a battery or an accumulator. The energy storage device 109 may be connected to the controller, the grid 108, and/or the UV-C emitter 106, and/or may provide power or electrical energy to one, more, or all of them. Alternatively, or additionally, the filter 100 may comprise a capacitor 120 that may be connected to the energy storage device 109. The capacitor 120 may be connected to the grid 108 and/or be configured to provide power to the grid 108 and/or serve to electrostatically charge the grid 108.

The filter 100 and/or the mask 1000 may comprise a data storage (memory) 110. The data storage 10 may be connected to the controller 111 such that the controller 111 may store data in or retrieve data from the data storage 110. The data may be, for example, control or regulation data, e.g., for controlling the UV emitter 106 or the grid 108. The data may also comprise, for example, information regarding a breathing rate, aerosols in the exhaled air, a composition of saliva or the like.

The filter 100 and/or the mask 1000 may comprise a telecommunications module (transceiver) 112. The telecommunications module 112 may be, for example, a radio interface. The telecommunications interface 112 may be configured to wirelessly receive data from or transmit data to external devices. The telecommunication module 112 may be connected to the data storage 110 and/or the controller 111 such that data may be exchanged between the telecommunication module 112 and data storage 110 and/or controller 111.

The filter 100 and/or mask 1000 may comprise an interface 113, for example a USB port for connecting a data transfer cable. The interface 113 may be configured to receive data from or transmit data to external devices via a cable. The interface 113 may be connected to the data storage 110 and/or the controller 111 such that data may be exchanged between the interface 113 and the data storage 110 and/or controller 111. Via the telecommunications module 112 and/or the interface 113 for example, control data may be communicated to the controller 111. The control data can specify or modify a control or regulation of the controller 111.

The filter 100 may comprise a sensor 101, 119. The sensor 101, 119 may be configured to detect aerosols or smoke, for example, in an environment of the mask and/or in the fluid flowing through the filter. For example, the sensor 101, 119 may be or comprise a temperature sensor or an ionization smoke detector, or measure heat, temperature, humidity, pressure, sound field quantities, brightness, accelerations, pH values, ionic strength, electrochemical potential, and/or material characteristics. The filter may comprise a fire alarm 103. The sensor 101, 119 may be a flow sensor and/or be configured to detect a through flow. For example, the sensor 101, 119 may be arranged at, near, or fluidically upstream of the grid 108, the UV emitter 106, between the first layer 118 and the second layer 117, and/or the filter fleece. The sensor 101, 119 may be connected to the controller 111 and/or the data storage 110 so that data measured by the sensor can be transmitted to the controller 111 and/or the data storage 110.

The filter 100 may comprise a solar cell 104. It may also be provided that alternatively or additionally the mask 1000 and/or the mask body 1 comprises an or the solar cell 104. The solar cell 104 may convert radiant energy, such as sunlight, into electrical energy. The solar cell 104 may be connected to the energy storage 109 or the energy storage receptacle 107, such that the energy storage 109 may be charged by the solar cell 104.

The filter 100 may comprise a holder 105. The holder 105 may be or comprise a frame or the like. The holder 105 may surround or enclose the filter 100 and stiffen the filter 100.

The filter 100 may comprise an actuating element 102 with which the controller 111 and/or functions of the filter and/or mask may be turned on and off. The actuating element 102 may be or comprise a button, a slider, a knob or the like.

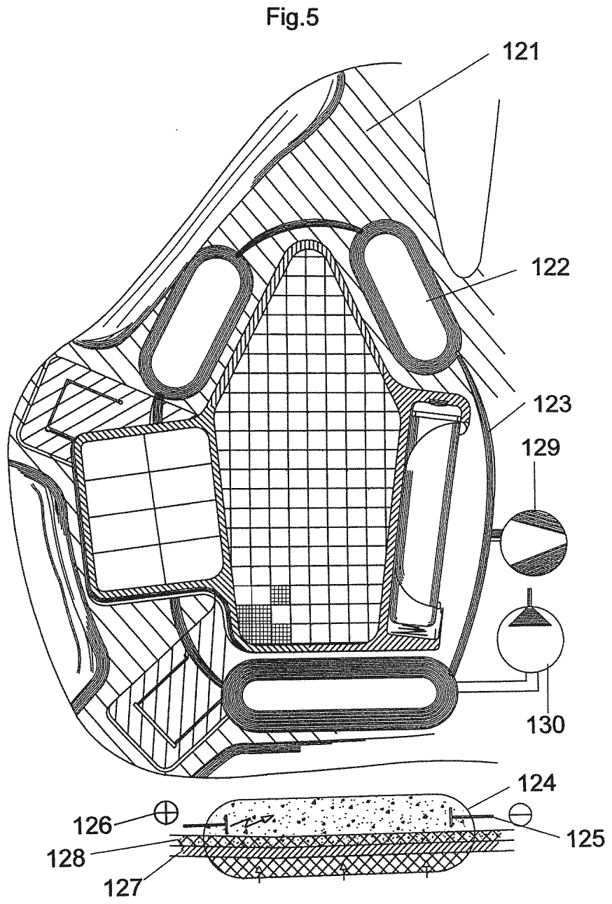

FIG. 5 shows an embodiment of a mask 1000. The mask 1000 and/or the mask body 1 can at least sectionally comprise a superabsorber. The superabsorber may be adapted to absorb liquid, for example condensate of exhaled air. Superabsorbers can absorb many times their own weight of polar liquids, for example water or aqueous solutions. When the liquid is absorbed, the superabsorbent swells and forms a hydrogel, whereby the sum of the volume of the liquid and the volume of the dry superabsorber remains the same.

The superabsorber may form at least a portion 121 of the mask body 1. The superabsorber can be arranged on an inner side of the mask. The inner side may mean the side facing a face of a mask wearer 1001 when the mask is put on. For example, the superabsorber may be arranged at a location where condensate collects or exhaled air condensates. For example, the superabsorber may be arranged in an area that may be at, near, or close to a mouth or nose region of the mask 1000 and/or mask wearer 1 in a donned state of the mask. The superabsorber may comprise a polymer, polyacrylamide, polyvinylpyrrolidone, amylopectin, gelatin, cellulose, and/or activated carbon, or combinations thereof. Alternatively, or additionally, the superabsorbent may comprise a molecular sieve, e.g., comprising zeolites. The molecular sieve may have, for example, a pore width of 3 Å. At this pore width, the molecular sieve can adsorb e.g., NH3 or H2O and/or be suitable for drying polar solvents. Alternatively, or additionally, the molecular sieve may have, for example, a pore width of 4 Å. At this pore width, the molecular sieve can adsorb e.g., H2O, CO2, SO2, H2S, C2H4, C2H6, C3H6, EtOH. Does not adsorb C3H8 and higher hydrocarbons and/or be suitable for drying apolar solvents and gases. Alternatively, or additionally, the molecular sieve may have, for example, a pore width of 5 Å. At this pore width, the molecular sieve can adsorb, for example, normal (linear) hydrocarbons up to n-C4H10, alcohols up to C4H9OH, mercaptans up to C4H9SH. Alternatively or additionally, the molecular sieve can have e.g., a pore width of 8 Å. At this pore width, the molecular sieve may adsorb, for example, branched hydrocarbons and aromatic compounds and/or be suitable for drying gases. Alternatively, or additionally, the molecular sieve may have, for example, a pore width of 10 Å. At this pore width, the molecular sieve can adsorb e.g., di-n-butylamine and/or be suitable for drying HMPT.

With the superabsorber moisture inside the mask can be reduced or completely prevented, so that the filter effect can be or is increased. For example, the electrostatic charge of the filter 100 can be maintained for longer because liquid and moisture are absorbed by the superabsorber. In addition, by reducing the moisture inside the mask, the wearing comfort can be or can get improved.

The mask 1000 may comprise one or more oxygen generators 122. With the oxygen generator 122 oxygen can be added to the fluid or air supplied to the mask wearer, or to the fluid or air flowing through the filter 100 and/or the mask 1000. If the mask 1000 comprises multiple oxygen generators 122, these can be fluidically connected to each other by means of one or more lines 23.

It can be provided to first pass the air supplied to the oxygen generator through a filter system to remove microorganisms and dust.

The oxygen generator 122 may generate oxygen using a pressure swing adsorption process. In one embodiment, special porous materials (e.g., zeolites or activated carbon) may be used as adsorbents. The separation effect can be achieved by means of different principles. In a first variant, the separation may occur due to, for example, an equilibrium adsorption. In a second variant, the separation can occur due to, for example, a molecular sieve effect. In the first case, one of the components to be separated may be more strongly adsorbed than another, whereby an enrichment of the more poorly adsorbed component in the gas phase may take place. In the second case, certain molecules can penetrate the porous structure of the adsorbent more quickly. If a gas mixture now flows through the adsorbent in a reactor bed, the component that penetrates the pores more poorly takes less time to flow past, hence reach the exit of the reactor bed sooner. This allows oxygen to be extracted from air supplied to the oxygen generator 122.

The oxygen generator 122 may be fluidically connected to an oxygen-admixing unit (not shown in the figures). The oxygen admixing unit may be fluidically connected to the filter 100. The oxygen admixing unit may be configured to mix the oxygen generated by the oxygen generator to the air filtered by the filter 100. The mixed air with the admixed oxygen can subsequently be driven out by the oxygen admixing unit from the mask 1000 towards the inside of the mask, i.e., supplied to the mask wearer 1001 when the mask 1000 is put on. In this case, it may be provided to also filter the air supplied to the oxygen generator, in particular to filter out pollutants and/or viruses with a further filter, e.g., a further filter 100.

However, it may also be provided that the oxygen admixing unit admixes the oxygen generated by the oxygen generator 122 to an ambient air that may for example flow into the oxygen admixing unit unfiltered, respectively enriches it with oxygen. In this case, it may be provided that the oxygen admixing unit is fluidically connected to the filter 100 so that the filter 100 filters the admixed air, e.g., viruses and/or pollutants. The admixed air filtered by the filter 100 can then be driven out by the oxygen admixing unit from the mask 1000 to the inside of the mask, i.e., supplied to the mask wearer 1001 when the mask 1000 is put on.

The oxygen generator 122 and/or its adsorbent must be regenerated from time to time, e.g., by driving out the adhering nitrogen. It may therefore be provided that the mask 1000 comprises a plurality of oxygen generators which alternately generate oxygen and are regenerated. In particular, it may be provided that always at least one oxygen generator 122 generates oxygen while at least one other oxygen generator 122 is regenerated.

Alternatively, or additionally, at least one of the oxygen generators 122 may obtain oxygen by electrolysis. The electrolysis may be a water electrolysis, in which water may be separated into hydrogen and oxygen. For this purpose, the oxygen generator 122 may have two electrodes respectively a cathode 125 and an anode 126. By applying electrical energy, the water can be separated into hydrogen and oxygen.

The oxygen generator 122 may be connected to and powered by the energy storage 109. The oxygen generator 122 can be connected to the controller 111 and can be controlled by it or receive control commands from it.

The oxygen generator 122 may comprise a sleeve 124. The sleeve 124 may be, for example, a fine mesh fabric sleeve. For example, the sleeve may be or comprise a microporous membrane of polytetrafluoroethylene.

The oxygen generator 122 may comprise a foil 127, which may be a semi-permeable foil and may prevent penetration into the interior of the mask. Through a supply line 128, a fluid, such as water, may be guided into the oxygen generator 122. The fluid may be or comprise, for example, condensate of exhaled air.

It may be provided that the filter 100 and/or the mask 1000 comprises a pump 129 for cleaning, for example, the filter 100, the grid 108, the first layer 118, the second layer 117, the filter fleece, the oxygen generator 122 and/or the oxygen admixing unit. The pump 129 may be arranged and/or fluidically connected accordingly.

It may be provided that the filter 100 and/or the mask 1000 comprises a compressor 130. The compressor 130 can, for example, support the build-up of pressure inside the mask during exhalation and/or support the pressure swing adsorption process.

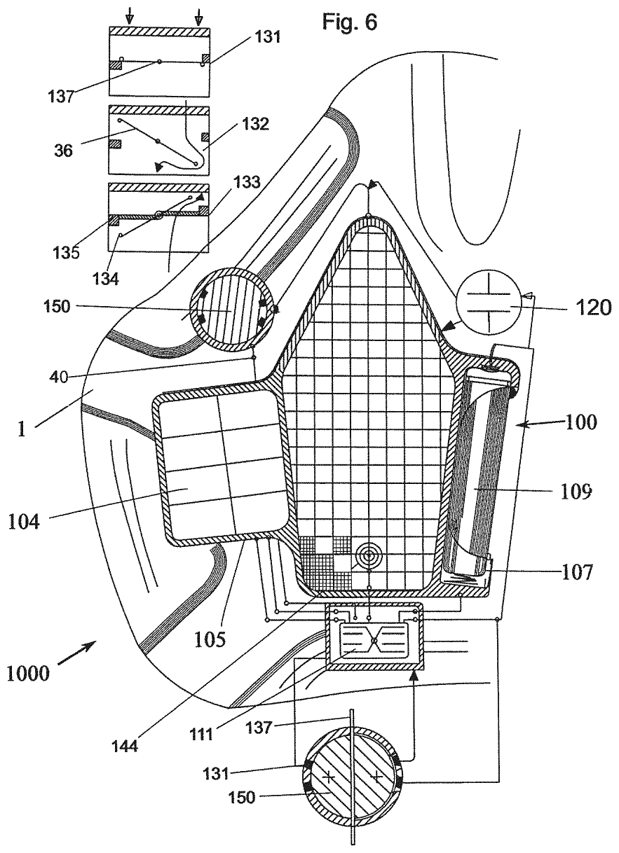

FIG. 6 shows an embodiment of a mask 1000 with a filter 100 described with reference to FIG. 4. The filter 100 and/or the mask 1000 may comprise a flow sensor 150. The sensor 101, 119 may be or comprise the flow sensor 150. The flow sensor may be arranged fluidically upstream of the filter 100 and/or the grid 108 or the filter fleece. The arrangement of the flow sensor 150 shown in FIG. 6 is merely exemplary. In the example embodiment shown in FIG. 6, for example, air may flow through the flow sensor 150 and be directed into the filter 100 through a line not shown in the figure. However, it may also be provided that the flow sensor 150 is arranged on, in, or near the filter 100 and/or the filter holder 6. For example, it may be provided that a fluid, such as air, flows directly from the environment into the filter 100 and/or the filter holder 6. The flow sensor 150 may be configured to detect a flow through the filter 100. The flow sensor may be configured to measure a flow velocity. The flow sensor 150 may be connected to the controller 111 so that measured values, signals, or data may be transmitted from the flow sensor 150 to the controller 111. The flow sensor may be fluidically connected to the filter 100 and/or the oxygen generator 122, such that fluid flowing through the flow sensor 150 flows from the flow sensor 150 into the filter 100 and/or the oxygen generator 122. The flow sensor 150 may be open to an environment of the mask 1000, such that fluid or air from the environment may flow into the flow sensor 150. The flow sensor 150 may be arranged on an outer side of the mask 1000. In the donned state of the mask, the outer side may correspond to the side facing away from the face of the mask wearer respectively the side opposite the inner side.

In one embodiment, the flow sensor 150 may include a pivot plate 136 pivotable about a rotation axis 137. The flow sensor 150 may have a contact point 131, a contact switch 133, a contact transmitter 134, and/or a contact receiver 135. If a fluid flows through the flow sensor 150, see for example streamline 132, the pivot plate 136 may be or become pivoted about the rotation axis 137. In a rest position, in which the flow sensor 150 does not have fluid flowing through it, it may be provided that the pivot plate 136 contacts the contact point 131. For this purpose, the pivot plate may comprise, for example, a contact transmitter 134 that may contact the contact point 131, for example, in the rest position. When the contact point 131 is contacted, the flow sensor 150 may communicate a non-flow-through to the controller 111. For example, a corresponding non-flow-through signal, such as a voltage or the like, may be communicated or applied to the controller 111 via the closed contact point 131. If the flow sensor 150 is flowed through, the pivot plate may be or become pivoted to a flow-through position in which the contact point 131 no longer is or gets contacted. In this case, the flow sensor 150 may communicate a flow-through to the controller 111. It may be provided that the flow sensor 150 communicates a flow-through signal to the controller 111. Alternatively, or additionally, it may be provided that the non-throughflow signal is not or does not get communicated to the controller 111, and/or the controller 111 may conclude a throughflow of the flow sensor 150 from the non-throughflow signal that is no longer communicated. Alternatively, or additionally, the flow sensor 150 may comprise one or more contact taker 135 arranged in such a way that the contact taker 135 may be or become contacted by the pivot plate 136 and/or the contact transmitter 134 in the flow-through position. However, the pivot plate 136, the contact switch 133, the contact point 131 and/or the contact taker 135 can also be arranged in such a way that the pivot plate 136 and/or the contact giver 134 can contact the contact taker 135 and/or cannot contact the contact point 131 in the rest position, and correspondingly cannot contact the contact taker 135 and/or contact the contact point 131 in the flow-through position. The flow direction through the flow sensor 150 may be suitably guided for this purpose.

However, other flow sensors are also conceivable or usable.

In one embodiment, the controller 111 may be configured to, upon a detected flow-through of the flow sensor 150, electrically switch or connect the solar cell 104 to the grid 108 and/or the UV emitter 106, or to close a corresponding switch or activate a switching element accordingly. The controller 111 may measure or detect the amount of energy provided by the solar cell 104. If the solar cell 104 does not provide sufficient electrical energy, the controller 111 may electrically switch the energy storage 109 with the grid 108 and/or the UV emitter 106 so that the grid 108 and/or the UV emitter 106 are powered by the energy storage 109. A sensor 144 may be provided that can measure the voltage or static charge applied to the grid 108 and/or UV emitter 106 and communicate it to the controller 111. If the voltage and/or static charge is too low, the controller 111 may electrically switch the capacitor 120 with the grid 108. However, the controller is not limited to the actuation described herein. It can be provided that with each breath the grid 108 is supplied with electrical power and/or a voltage is applied.

It may be provided that the solar cell 104 can only charge the energy storage 109 and/or the capacitor 120 and is not used to directly power the grid 108 and/or the UV emitter 106.

Alternatively, or additionally, it may be provided that the grid 108 and/or the UV emitter 106 is supplied with electric current or voltage only at predetermined, e.g. fixed, time intervals for a predetermined duration. The controller 111 may be suitably configured for this purpose respectively switch or control suitably. The time interval and/or duration may depend on the voltage measured by the sensor 144. It is apparent that other controls are possible.

The features disclosed in the description, figures and claims may be essential to the disclosure individually or in any combination.

To enable those skilled in the art to better understand the solution of the present disclosure, the technical solution in the embodiments of the present disclosure is described clearly and completely below in conjunction with the drawings in the embodiments of the present disclosure. Obviously, the embodiments described are only some, not all, of the embodiments of the present disclosure. All other embodiments obtained by those skilled in the art on the basis of the embodiments in the present disclosure without any creative effort should fall within the scope of protection of the present disclosure.

It should be noted that the terms “first”, “second”, etc. in the description, claims and abovementioned drawings of the present disclosure are used to distinguish between similar objects, but not necessarily used to describe a specific order or sequence. It should be understood that data used in this way can be interchanged as appropriate so that the embodiments of the present disclosure described here can be implemented in an order other than those shown or described here. In addition, the terms “comprise” and “have” and any variants thereof are intended to cover non-exclusive inclusion. For example, a process, method, system, product or equipment comprising a series of steps or modules or units is not necessarily limited to those steps or modules or units which are clearly listed, but may comprise other steps or modules or units which are not clearly listed or are intrinsic to such processes, methods, products or equipment.

References in the specification to “one embodiment,” “an embodiment,” “an exemplary embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The exemplary embodiments described herein are provided for illustrative purposes, and are not limiting. Other exemplary embodiments are possible, and modifications may be made to the exemplary embodiments. Therefore, the specification is not meant to limit the disclosure. Rather, the scope of the disclosure is defined only in accordance with the following claims and their equivalents.

### Reference List

1 mask body

2 first zone

3 shaper

4 closure

6 filter holder

7 mounting interface

8 reinforced zone

10 flexible zone

51 reinforcement zone

52 holder

53 mask attachment

54 connecting element

101 sensor

102 actuating element

103 fire alarm

104 solar cell

105 holder

106 UV-emitter

107 energy storage receptable

108 grid

109 energy storage

110 data storage (memory)

111 controller

112 telecommunications module

113 interface

114 layer

115 molecular sieve

117 second layer

118 first layer

119 sensor

120 capacitor

121 mask body portion

122 oxygen generator

124 sleeve

125 cathode

126 anode

127 foil

128 supply line

129 pump

130 compressor

131 contact point

132 streamline

133 contact switch

134 contact transmitter

135 contact taker

136 pivot plate

137 rotation axis

144 sensor

1000 mask

1001 mask wearer

1002 cut out

1003 strap

1005 slip-on attachment

1. A respirator mask comprising:
a mask body; and
at least one filter configured to filter a fluid flowing through the respirator mask and/or the mask body, wherein the at least one filter includes: an ultra-violet (UV) filter and/or UV emitter, filter fleece, and/or an electrical and/or electrostatic filter.

2. The respirator mask according to claim 1, further comprises at least one filter holder configured to receive and hold the at least one filter, wherein the at least one filter holder is arranged at least in a mouth region and/or in a nose region of a mask wearer in a donned state of the respirator mask.

3. The respirator mask according to claim 1, wherein the at least one filter is detachably connected to the respirator mask and/or the mask body.

4. The respirator mask according to claim 1, wherein the at least one filter comprises a metal grid including copper, brass, silver, and/or gold.

5. The respirator mask according to claim 4, wherein the at least one filter further comprises first and second layers, the grid being arranged between the first layer and the second layer, wherein the first layer and/or the second layer comprises a fleece and/or the filter fleece.

6. The respirator mask according to claim 5, wherein the first layer and/or the second layer is or comprises a meltblown fleece, the meltblown fleece including a plastic.

7. The respirator mask according to claim 1, wherein the at least one filter comprises a molecular sieve including zeolites or activated carbon.

8. The respirator mask according to claim 1, wherein the UV emitter is arranged in the at least one filter such that the filter fleece is illuminable by the UV emitter, the filter fleece including a fluorescent substance configured to be activated by the UV emitter.

9. The respirator mask according to claim 1, wherein the respirator mask and/or the at least one filter comprises an energy storage and a controller, the energy storage being:
connected to the electrical filter and/or the grid for power supply, wherein the controller is configured to control or regulate the power supply, and/or
connected to the UV emitter for power supply, the controller being configured to control or actuate the UV emitter.

10. The respirator mask according to claim 9, wherein the respirator mask and/or the at least one filter comprises a solar cell connected to the energy storage and/or the controller, such that the energy storage is configured to be chargeable by the solar cell.

11. The respirator mask according to claim 1, wherein the respirator mask and/or the at least one filter comprises a flow sensor configured to detect: (a) fluid to be filtered flowing in through the mask and/or the at least one filter, and/or (b) fluid flowing out of the mask and/or the at least one filter.

12. The respirator mask according to claim 11, wherein the flow sensor is connected a controller, such that, upon detection of fluid flowing through the respirator mask and/or the at least one filter, the controller is configured to:
control a power supply to a grid of the at least one filter and/or the electrical filter; and/or
control the UV emitter to illuminate the filter fleece.

13. The respirator mask according to claim 11, wherein the flow sensor comprises a pivot plate and a contact which is pivotable about an axis of rotation into a flow-through position when flow passes through the at least one filter, such that in the flow-through position, the pivot plate contacts the contact.

14. The respirator mask according to claim 1, wherein the mask body comprises, at least sectionally, a superabsorber forming at least part of the mask body.

15. The respirator mask according to claim 14, wherein the superabsorber is arranged on or in an inner side of the respirator mask such that the superabsorber is configured to absorb moisture located inside the respirator mask in a donned state of the respirator mask.

16. The respirator mask according to claim 1, wherein the respirator mask comprises an oxygen generator fluidically connected to an oxygen-admixing unit configured to enrich fluid flowing through the respirator mask with oxygen.

17. The respirator mask according to claim 16, wherein the oxygen generator is configured to provide or generate oxygen using a pressure swing adsorption process or electrolysis.

18. The respirator mask according to claim 16, the respirator filter is fluidically connected to the oxygen-admixing unit, such that air entering the respirator mask is filtered by the filter and supplied to the oxygen-admixing unit.
